(19) United States
(12) Patent Application Publication
(10) Pub. No.: US 2026/0306472 A1
(43) Pub. Date: Oct. 8, 2026

(54) PYRIDYLAMINOACETIC ACID COMPOUND AND POLYOXYETHYLENE CASTOR OIL-CONTAINING PHARMACEUTICAL COMPOSITION

(71) Applicant: Santen Pharmaceutical Co., Ltd., Osaka (JP)

(72) Inventor: Yoko ENDO, Ikoma-shi (JP)

(21) Appl. No.: 19/703,755

(22) Filed: Jun. 10, 2026

Related U.S. Application Data
(63) Continuation of application No. 18/363,961, filed on Aug. 2, 2023, now Pat. No. 12,678,429.
Application No. 18/363,961 is a continuation of application No. 16/711,706, filed on Dec. 12, 2019, now abandoned.
Application No. 16/711,706 is a continuation of application No. 15/204,475, filed on Jul. 7, 2016, now abandoned.
Application No. 15/204,475 is a continuation of application No. PCT/JP2015/050333, filed on Jan. 8, 2015.

(30) Foreign Application Priority Data
Jan. 10, 2014 (JP) 2014-002809

(51) Int. Cl. A61K 31/444; A61K 9/00; A61K 9/08; A61K 47/02; A61K 47/12; A61K 47/18; A61K 47/44

(52) U.S. Cl. CPC A61K 31/444; A61K 9/0048; A61K 9/08; A61K 47/02; A61K 47/12; A61K 47/183; A61K 47/44; Y02A 50/30

(57) ABSTRACT

The purpose of the present invention is to provide: a pharmaceutical composition that comprises the pyridylaminoacetic acid compound of the present invention, said pyridylaminoacetic acid compound being stable within the pharmaceutical composition; and a method for improving the stability of the pydridylaminoacetic acid compound within the pharmaceutical composition. The pharmaceutical composition comprises: (6{[4-(pyrazol-1-yl)benzyl](pyridine-3-ylsulfonyl)aminomethyl}pyridine-2-ylamino)isopropyl acetate or a salt thereof; and polyoxyethylene castor oil. The polyoxyethylene castor oil preferably comprises a polyoxyethylene castor oil selected from the group consisting of polyoxyl 5 castor oil, polyoxyl 9 castor oil, polyoxyl 15 castor oil, polyoxyl 35 castor oil, and polyoxyl 40 castor oil.

### CROSS REFERENCE TO RELATED APPLICATIONS

This application is a continuation of U.S. patent application Ser. No. 18/363,961, filed Aug. 2, 2023. U.S. Patent application No. 18,363,961 is a continuation of U.S. patent application Ser. No. 16/711,706, filed on Dec. 12, 2019, now abandoned. U.S. patent application Ser. No. 16/711,706 is a continuation of Ser. No. 15/204,475, filed on Jul. 7, 2016, now abandoned. U.S. patent application Ser. No. 15/204,475 is a continuation of PCT/JP2015/050333, filed on Jan. 8, 2015, and which claims priority to and the benefit of Japanese Application No. 2014-002809, filed Jan. 10, 2014. The contents of each of the above is incorporated by reference herein in their entirety for all purposes.

### TECHNICAL FIELD

The present invention relates to a pharmaceutical composition comprising Isopropyl(6-{[4-(pyrazol-1-yl)benzyl](pyridin-3-ylsulfonyl)aminomethyl}pyridin-2-ylamino)acetate or a salt thereof, and a method for stabilizing the compound or salt thereof.

### BACKGROUND ART

Isopropyl(6-{[4-(pyrazol-1-yl)benzyl](pyridin-3ylsulfonyl)aminomethyl}pyridin-2-ylamino)acetate is a compound represented by the following formula (1):

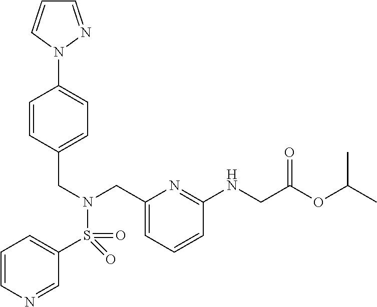 [SMILES: CC(C)OC(=O)CNc1cccc(CN(Cc2ccc(-n3cccn3)cc2)S(=O)(=O)c2cccnc2)n1]

Patent Document 1 and Patent Document 2 mention pyridylaminoacetic acid compounds such as isopropyl (6-{[4-(pyrazol-1-yl)benzyl](pyridin-3-ylsulfonyl)aminomethyl}pyridin-2-ylamino)acetate, and Patent Document 1 mentions, as eye drops of the pyridylaminoacetic acid compound, Formulation Examples comprising concentrated glycerol and Polysorbate 80.

However, Patent Document 1 does not mention a pharmaceutical composition comprising isopropyl (6-{[4-(pyrazol-1-yl)benzyl](pyridin-3-ylsulfonyl)aminomethyl}pyridin-2-ylamino)acetate or a salt thereof and polyoxyethylene castor oil, and also there is absolutely no mention that polyoxyethylene castor oil improves stability of the isopropyl (6-{[4-(pyrazol-1-yl)benzyl](pyridin-3-ylsulfonyl)aminomethyl}pyridin-2-ylamino)acetate or salt thereof in the pharmaceutical composition.
Patent Document 1: U.S. Published Patent Application Publication, No. 2012/0190852, Specification
Patent Document 2: U.S. Published Patent Application Publication, No. 2011/0054172, Specification

### DISCLOSURE OF THE INVENTION

### Problems to be Solved by the Invention

At a stage of development of a pharmaceutical composition comprising isopropyl (6-{[4-(pyrazol-1-yl)benzyl](pyridin-3-ylsulfonyl)aminomethyl}pyridin-2-ylamino)acetate or a salt thereof (hereinafter also referred to as “the present compound”), the present inventors have found that, in an aqueous composition comprising the present compound dissolved therein, stability of the present compound is inferior.

An object of the present invention is to provide a pharmaceutical composition comprising the present compound, in which the present compound in the pharmaceutical composition is stable. Another object of the present invention is to provide a method for improving stability of the present compound in the pharmaceutical composition.

### Means for Solving the Problems

The present inventors have intensively studied about a surfactant for dissolving the present compound so as to achieve the above objects, and found that the present compound in a pharmaceutical composition has a high remaining rate even under long-term storage when using polyoxyethylene castor oil, thus completing the present invention. Namely, the present invention is related to the following.
(1) A pharmaceutical composition comprising isopropyl (6-{[4-(pyrazol-1-yl)benzyl](pyridin-3-ylsulfonyl)aminomethyl}pyridin-2-ylamino)acetate or a salt thereof, and polyoxyethylene castor oil.
(2) The pharmaceutical composition according to (1), wherein the polyoxyethylene castor oil includes polyoxyethylene castor oil selected from the group consisting of polyoxyl 5 castor oil, polyoxyl 9 castor oil, polyoxyl 15 castor oil, polyoxyl 35 castor oil, and polyoxyl 40 castor oil.
(3) The pharmaceutical composition according to (2), wherein the polyoxyethylene castor oil include polyoxyl 35 castor oil.
(4) The pharmaceutical composition according to any one of (1) to (3), wherein the content of 6-{[4-(pyrazol-1-yl)benzyl](pyridin-3-ylsulfonyl)aminomethyl}pyridin-2-ylamino)isopropyl acetate or a salt thereof is in a range of 0.0001 to 0.1% (w/v).
(5) The pharmaceutical composition according to (4), wherein the content of 6-{[4-(pyrazol-1-yl)benzyl](pyridin-3-ylsulfonyl)aminomethyl}pyridin-2-ylamino)isopropyl acetate or a salt thereof is in a range of 0.001 to 0.003% (w/v).
(6) The pharmaceutical composition according to any one of (1) to (5), wherein the content of the polyoxyethylene castor oil is in a range of 0.001 to 5% (w/v).
(7) The pharmaceutical composition according to (6), wherein the content of the polyoxyethylene castor oil is in a range of 0.8 to 2% (w/v).
(8) The pharmaceutical composition according to any one of (1) to (7), wherein the content of the polyoxyethylene castor oil is in a range of 1 to 20,000 parts by mass relative to 1 part by mass of 6-{[4-(pyrazol-1-yl)benzyl](pyridin-3-ylsulfonyl)aminomethyl}pyridin-2-ylamino)isopropyl acetate or a salt thereof.
(9) The pharmaceutical composition according to (8), wherein the content of the polyoxyethylene castor oil is in a range of 200 to 2,000 parts by mass relative to 1 part by mass of 6-{[4-(pyrazol-1-yl)benzyl](pyridin-3-ylsulfonyl)aminomethyl}pyridin-2-ylamino)isopropyl acetate or a salt thereof.
(10) The pharmaceutical composition according to any one of (1) to (9), which further comprises edetic acid or a salt thereof.
(11) The pharmaceutical composition according to any one of (1) to (10), which further comprises boric acid or a salt thereof, citric acid or a salt thereof, or acetic acid or a salt thereof.
(12) The pharmaceutical composition according to any one of (1) to (11), which does not comprise sorbic acid.
(13) The pharmaceutical composition according to any one of (1) to (12), which is filled into a container made of polyethylene.
(14) The pharmaceutical composition according to any one of (1) to (13), for prevention or treatment of glaucoma or ocular hypertension, or for reduction of intraocular pressure.
(15) A method for stabilizing isopropyl (6-{[4-(pyrazol-1-yl)benzyl](pyridin-3-ylsulfonyl)aminomethyl}pyridin-2-ylamino)acetate or a salt thereof by allowing a pharmaceutical composition comprising isopropyl (6-{[4-(pyrazol-1-yl)benzyl](pyridin-3-ylsulfonyl)aminomethyl}pyridin-2-ylamino)acetate or a salt thereof to comprise polyoxyethylene castor oil.

The respective structures of the above-mentioned (1) to (15) can be combined by optionally selecting two or more structures therefrom.

### Effects of the Invention

According to the present invention, it is possible to provide a pharmaceutical composition in which the present compound in the pharmaceutical composition is stabilized over a long period of time. The pharmaceutical composition of the present invention has enough safety as a pharmaceutical product. According to the present invention, it is also possible to provide a method for stabilizing the present compound in a pharmaceutical composition over a long period of time. According to the present invention, it is also possible to provide a method for using polyoxyethylene castor oil so as to produce a pharmaceutical composition in which the present compound in the pharmaceutical composition is stabilized over a long period of time.

### PREFERRED MODE FOR CARRYING OUT THE INVENTION

Embodiments of the present invention will be described in detail below.

It is possible to produce isopropyl (6-{[4-(pyrazol-1-yl)benzyl](pyridin-3-ylsulfonyl)aminomethyl}pyridin-2-ylamino)acetate or a salt thereof comprised in a pharmaceutical composition of the present invention in accordance with a conventional method in the technical field, such as a method mentioned in U.S. Published Patent Application Publication, No. 2012/0190852, Specification.

In the pharmaceutical composition of the present invention, a salt of isopropyl (6-{[4-(pyrazol-1-yl)benzyl](pyridin-3-ylsulfonyl)aminomethyl}pyridin-2-ylamino)acetate is not particularly limited as long as it is a pharmacologically acceptable salt. Specifically, there are exemplified inorganic acid salts such as hydrochlorides, hydrobromates, hydroiodides, nitrates, sulfates, or phosphates; or organic acid salts such as acetates, trifluoroacetates, benzoates, oxalates, malonates, succinates, maleates, fumarates, tartrates, citrates, methanesulfonates, ethanesulfonates, trifluoromethanesulfonates, benzenesulfonates, p-toluenesulfonates, glutamates, or aspartates. Preferably, hydrochlorides or trifluoroacetates are exemplified.

In the pharmaceutical composition of the present invention, the content of isopropyl(6-{[4-(pyrazol-1-yl)benzyl](pyridin-3-ylsulfonyl)aminomethyl}pyridin-2-ylamino)acetate or a salt thereof is not particularly limited. Specifically, the lower limit is preferably 0.0001% (w/v), more preferably 0.0003% (w/v), still more preferably 0.0005% (w/v), and yet still more preferably 0.001% (w/v). The upper limit is preferably 0.1% (w/v), more preferably 0.03% (w/v), still more preferably 0.01% (w/v), yet still more preferably 0.008% (w/v), even still more preferably 0.005% (w/v), and particularly preferably 0.003% (w/v). More specifically, the content is preferably in a range of 0.0001 to 0.1% (w/v), more preferably 0.0003 to 0.03% (w/v), still more preferably 0.0005 to 0.01% (w/v), yet still more preferably 0.001 to 0.008% (w/v), even still more preferably 0.001 to 0.005% (w/v), and particularly preferably 0.001 to 0.003% (w/v). Comparatively small content of the present compound may enable a reduction in amount of a surfactant (typically polyoxyethylene castor oil), which is required to dissolve the present compound, so that the content of the present compound is preferably less than 0.01% (w/v). When comprising a salt of isopropyl (6-{[4-(pyrazol-1-yl)benzyl](pyridin-3-ylsulfonyl)aminomethyl}pyridin-2-ylamino)acetate, it means that the content of isopropyl (6-{[4-(pyrazol-1-yl)benzyl](pyridin-3-ylsulfonyl)aminomethyl}pyridin-2-ylamino)acetate falls in the above range in a state where the salt is isolated.

In the pharmaceutical composition of the present invention, it is possible to use, as the polyoxyethylene castor oil, various polyoxyethylene castor oils each exhibiting different number of polymerization of ethylene oxide. The number of polymerization of ethylene oxide is preferably in a range of 5 to 100, more preferably 20 to 50, particularly preferably 30 to 40, and most preferably 35. Specific examples of the polyoxyethylene castor oil include polyoxyl 5 castor oil, polyoxyl 9 castor oil, polyoxyl 15 castor oil, polyoxyl 35 castor oil, polyoxyl 40 castor oil, and the like, and polyoxyl 35 castor oil is most preferable. The polyoxyethylene castor oil as used herein is not polyoxyethylene hardened castor oil (provided that it is not excluded from the present invention that the pharmaceutical composition of the present invention further comprises polyoxyethylene hardened castor oil in the amount which does not exert an adverse influence on stability).

In the pharmaceutical composition of the present invention, the content of the polyoxyethylene castor oil is not particularly limited. Specifically, the lower limit is preferably 0.001% (w/v), more preferably 0.01% (w/v), still more preferably 0.1% (w/v), particularly preferably 0.5% (w/v), and most preferably 0.8% (w/v). The upper limit is preferably 10% (w/v), more preferably 5% (w/v), still more preferably 4% (w/v), particularly preferably 3% (w/v), and most preferably 2% (w/v). More specifically, the content is preferably in a range of 0.001 to 10% (w/v), more preferably 0.01 to 5% (w/v), still more preferably 0.1 to 4% (w/v), particularly preferably 0.5 to 3% (w/v), and most preferably 0.8 to 2% (w/v).

In the pharmaceutical composition of the present invention, the content of the polyoxyethylene castor oil relative to 6-{[4-(pyrazol-1-yl)benzyl](pyridin-3-ylsulfonyl)aminomethyl}pyridin-2-ylamino)isopropyl acetate or a salt thereof is not particularly limited. Specifically, the lower limit of the content of the polyoxyethylene castor oil is preferably 1 part by mass, more preferably 10 parts by mass, still more preferably 50 parts by mass, yet still more preferably 100 parts by mass, and particularly preferably 200 parts by mass, relative to 1 part by mass of 6-{[4-(pyrazol-1-yl)benzyl](pyridin-3-ylsulfonyl)aminomethyl}pyridin-2-ylamino)isopropyl acetate or a salt thereof. The upper limit is preferably 20,000 parts by mass, more preferably 10,000 parts by mass, still more preferably 5,000 parts by mass, yet still more preferably 3,000 parts by mass, and particularly preferably 2,000 parts by mass. More specifically, the content of the polyoxyethylene castor oil is preferably in a range of 1 to 20,000 parts by mass, more preferably 10 to 10,000 parts by mass, still more preferably 50 to 5,000 parts by mass, particularly preferably 100 to 3,000 parts by mass, and most preferably 200 to 2,000 parts by mass, relative to 1 part by mass of 6-{[4-(pyrazol-1-yl)benzyl](pyridin-3-ylsulfonyl)aminomethyl}pyridin-2-ylamino)isopropyl acetate or a salt thereof.

Additives can be optionally used in the pharmaceutical composition of the present invention, and it is possible to add, as additives, a buffer agent, a tonicity agent, a stabilizer, a preservative, an antioxidant, a high molecular weight polymer, and the like.

It is possible to mix the buffer agent, which is usable as additives for a pharmaceutical product, in the pharmaceutical composition of the present invention. Examples of the buffer agent include phosphoric acid or a salt thereof, boric acid or a salt thereof, citric acid or a salt thereof, acetic acid or a salt thereof, carbonic acid or a salt thereof, tartaric acid or a salt thereof, F-aminocaproic acid, trometamol, and the like. From a viewpoint of buffering capacity in a weak acid region, the buffer agent is preferably boric acid or a salt thereof, citric acid or a salt thereof, or acetic acid or a salt thereof, and particularly preferably citric acid or a salt thereof. Examples of the phosphate include sodium phosphate, sodium dihydrogen phosphate, disodium hydrogen phosphate, potassium phosphate, potassium dihydrogen phosphate, dipotassium hydrogen phosphate, and the like; examples of the borate include borax, sodium borate, potassium borate, and the like; examples of the citrate include sodium acetate, disodium citrate, trisodium citrate, and the like; examples of the acetate include sodium acetate, potassium acetate, and the like; examples of the carbonate include sodium carbonate, sodium hydrogen carbonate, and the like; and examples of the tartrate include sodium tartrate, potassium tartrate, and the like. When the buffer agent is mixed in the pharmaceutical composition of the present invention, the content of the buffer agent can be appropriately adjusted according to the type of the buffer agent, and is preferably in a range of 0.001 to 10% (w/v), more preferably 0.01 to 5% (w/v), still more preferably 0.1 to 3% (w/v), and most preferably 0.2 to 2% (w/v).

It is possible to appropriately mix the tonicity agent, which is usable as additives for a pharmaceutical product, in the pharmaceutical composition of the present invention. Examples of the tonicity agent include an ionic tonicity agent, a nonionic tonicity agent, and the like. Examples of the ionic tonicity agent include sodium chloride, potassium chloride, calcium chloride, magnesium chloride, and the like; and examples of the nonionic tonicity agent include glycerol, propylene glycol, sorbitol, mannitol, and the like. When the tonicity agent is mixed in the pharmaceutical composition of the present invention, the content of the tonicity agent can be appropriately adjusted according to the type of the tonicity agent, and is preferably in a range of 0.01 to 10% (w/v), more preferably 0.02 to 7% (w/v), still more preferably 0.1 to 5% (w/v), particularly preferably 0.5 to 4% (w/v), and most preferably 0.8 to 3% (w/v).

It is possible to appropriately mix the stabilizer, which is usable as additives for a pharmaceutical product, in the pharmaceutical composition of the present invention. Examples of the stabilizer include edetic acid, monosodium edetate, disodium edetate, tetrasodium edetate, sodium citrate, and the like, and disodium edetate is particularly preferable. When the stabilizer is mixed in the pharmaceutical composition of the present invention, the content of the stabilizer can be appropriately adjusted according to the type of the stabilizer, and is preferably in a range of 0.001 to 1% (w/v), more preferably 0.005 to 0.5% (w/v), still more preferably 0.01 to 0.1% (w/v), and most preferably 0.02 to 0.05% (w/v).

It is possible to appropriately mix the preservative, which is usable as additives for a pharmaceutical product, in the pharmaceutical composition of the present invention. Examples of the preservative include benzalkonium chloride, benzalkonium bromide, benzethonium chloride, sorbic acid, potassium sorbate, methyl paraoxybenzoate, propyl paraoxybenzoate, chlorobutanol, and the like. From a viewpoint of stability of 6-{[4-(pyrazol-1-yl)benzyl](pyridin-3-ylsulfonyl)aminomethyl}pyridin-2-ylamino)isopropyl acetate or a salt thereof, it is desired not to include sorbic acid. When the preservative is mixed in the pharmaceutical composition of the present invention, the content of the preservative can be appropriately adjusted according to the type of the preservative, and is preferably in a range of 0.0001 to 1% (w/v), more preferably 0.0005 to 0.1% (w/v), still more preferably 0.001 to 0.05% (w/v), and most preferably 0.002 to 0.01% (w/v).

It is possible to appropriately mix the antioxidant, which is usable as additives for a pharmaceutical product, in the pharmaceutical composition of the present invention. Examples of the antioxidant include ascorbic acid, tocopherol, dibutylhydroxytoluene, butylhydroxyanisole, sodium erythorbate, propyl gallate, sodium sulfite, and the like. When the antioxidant is mixed in the pharmaceutical composition of the present invention, the content of the antioxidant can be appropriately adjusted according to the type of the antioxidant, and is preferably in a range of 0.0001 to 1% (w/v), more preferably 0.0005 to 0.1% (w/v), still more preferably 0.001 to 0.02% (w/v), and most preferably 0.005 to 0.010% (w/v).

It is possible to appropriately mix the high molecular weight polymer, which is usable as additives for a pharmaceutical product, in the pharmaceutical composition of the present invention. Examples of the high molecular weight polymer include methyl cellulose, ethyl cellulose, hydroxymethyl cellulose, hydroxyethyl cellulose, hydroxypropyl cellulose, hydroxyethylmethyl cellulose, hydroxypropylmethyl cellulose, carboxymethyl cellulose, sodium carboxymethyl cellulose, hydroxypropylmethylcellulose acetate succinate, hydroxypropylmethylcellulose phthalate, carboxymethylethyl cellulose, cellulose acetate phthalate, polyvinylpyrrolidone, polyvinyl alcohol, carboxyvinyl polymer, polyethylene glycol, and the like. When the high molecular weight polymer is mixed in the pharmaceutical composition of the present invention, the content of the high molecular weight polymer can be appropriately adjusted according to the type of the high molecular weight polymer, and is preferably in a range of 0.001 to 5% (w/v), more preferably 0.01 to 1% (w/v), and still more preferably 0.1 to 0.5% (w/v).

The pH of the pharmaceutical composition of the present invention is preferably in a range of 4.0 to 8.0, more preferably 4.5 to 7.5, and most preferably 5.0 to 7.0.

The pharmaceutical composition of the present invention can be stored in a container made of various raw materials. For example, it is possible to use containers made of polyethylene, polypropylene, and the like. From a viewpoint of ease of instillation (hardness of container) and stability of the present compound, it is preferred to store in a container made of polyethylene.

The dosage form of the pharmaceutical composition of the present invention is not particularly limited as long as it is usable as a pharmaceutical product. Examples of the dosage form include eye drop, ophthalmic injection, and the like, and eye drop is particularly preferable. They can be produced in accordance with a conventional method in the technical field. The pharmaceutical composition of the present invention is basically a solution, and a solvent or dispersion medium thereof is preferably water.

The pharmaceutical composition of the present invention is useful for prevention or treatment of glaucoma or ocular hypertension, or for reduction of intraocular pressure. Examples of glaucoma in the present invention include primary open-angle glaucoma, secondary open-angle glaucoma, normal tension glaucoma, hypersecretion glaucoma, primary closed-angle glaucoma, secondary closed-angle glaucoma, plateau iris glaucoma, mixed glaucoma, developmental glaucoma, steroid glaucoma, exfoliation glaucoma, amyloid glaucoma, neovascular glaucoma, malignant glaucoma, capsular glaucoma, plateau iris syndrome, and the like.

The pharmaceutical composition of the present invention may comprise one or plurality of, preferably 1 to 3 of, and more preferably one or two other glaucoma or ocular hypertension therapeutic agent(s) or intraocular tension depressor(s). The other glaucoma therapeutic agents is not particularly limited. Specifically, the other glaucoma therapeutic agent is preferably a commercially available glaucoma therapeutic agent or a glaucoma therapeutic agent under development, more preferably a commercially available glaucoma therapeutic agent, and particularly preferably a commercially available glaucoma therapeutic agent whose mechanism of action is different from that of the present compound. More specifically, there are exemplified a non-selective sympathomimetic agent, an α2 receptor agonist, an al receptor antagonist, a β receptor antagonist, a parasympatholytic agent, a carbonate dehydratase inhibitor, prostaglandins, a Rho kinase inhibitor, and the like. Specific examples of the non-selective sympathomimetic agent include dipivefrin; specific examples of the α2 receptor agonist include brimonidine and apraclonidine; specific examples of the al receptor antagonist include bunazosin; specific examples of the β receptor antagonist include timolol, befunolol, carteolol, nipradilol, betaxolol, levobunolol, and metipranolol; specific examples of the parasympatholytic agent include pilocarpine; specific examples of the carbonate dehydratase inhibitor include dorzolamide, brinzolamide, and acetazolamide; specific examples of prostaglandins include latanoprost, isopropyl unoprostone, bimatoprost, and travoprost; and specific examples of the Rho kinase inhibitor include ripasudil.

### EXAMPLES

Formulation Examples and test results will be shown below, but such are for better understanding of the present invention and do not limit the scope of the present invention.

### Formulation Examples

Typical Formulation Examples using the present compound will be shown below. In the following Formulation Examples, the mixing amount of each component is the content in 100 mL of the composition.

### Formulation Example 1

Eye drop (in 100 mL)
Present compound 0.001 g
Boric acid 0.2 g
Glycerol 2.0 g
Polyoxyl 35 castor oil 0.5 g
Disodium edetate 0.05 g
Benzalkonium chloride 0.005 g
Dilute hydrochloric acid q.s.
Sodium hydroxide q.s.
Purified water q.s.

### Formulation Example 2

Eye drop (in 100 mL)
Present compound 0.001 g
Sodium dihydrogen phosphate 0.2 g
Glycerol 2.0 g
Polyoxyl 35 castor oil 0.8 g
Disodium edetate 0.05 g
Benzalkonium chloride 0.005 g
Dilute hydrochloric acid q.s.
Sodium hydroxide q.s.
Purified water q.s.

### Formulation Example 3

Eye drop (in 100 mL)
Present compound 0.001 g
Trisodium citrate 0.2 g
Glycerol 2.0 g
Polyoxyl 35 castor oil 0.3 g
Disodium edetate 0.05 g
Benzalkonium chloride 0.005 g
Dilute hydrochloric acid q.s.
Sodium hydroxide q.s.
Purified water q.s.

Types and mixing amounts of the present compound, polyoxyethylene castor oil, and additives in Formulation Examples 1 to 3 can be appropriately adjusted to obtain desired compositions.

### 1. Stability Evaluation Test (1)

An influence of a surfactant on stability of the present compound was studied.

### 1-1. Preparation of Test Formulation

To 5 g of polyoxyl 35 castor oil, 20 mL of a 10% sodium dihydrogen phosphate solution and 900 mL of purified water were added. After adjusting the pH to about 6 by adding a sodium hydroxide solution or dilute hydrochloric acid (q.s.), 0.003 g of isopropyl (6-{[4-(pyrazol-1-yl)benzyl](pyridin-3-ylsulfonyl)aminomethyl}pyridin-2-ylamino)acetate (hereinafter also referred to as the compound A) was added and dissolved. To this was added purified water (q.s.) to make 1,000 mL in total, thus preparing a formulation of Example 1.

In the same manner as in preparation method of Example 1, formulations of Comparative Examples 1 and 2 shown in Table 1 were prepared.

### 1-2. Test Procedure

After filling a glass ampule with 5 mL of a test formulation and storing at 60° C. for an optional period, the content of isopropyl (6-{[4-(pyrazol-1-yl)benzyl](pyridin-3-ylsulfonyl)aminomethyl}pyridin-2-ylamino)acetate was determined using high-performance liquid chromatography, and then a remaining rate (%) thereof was calculated.

### 1-3. Test Results and Consideration

Test results are shown in Table 1.

TABLE 1

| | | Example | Comparative | Comparative |
|---|---|---|---|---|
| % (w/v) | | 1 | Example 1 | Example 2 |
| Present compound A | | 0.0003 | 0.0003 | 0.0003 |
| Polyoxyl 35 castor oil | | 0.5 | — | — |
| Polyoxyethylene hardened | | — | 0.5 | — |
| castor oil 60 | | | | |
| Polysorbate 80 | | — | — | 0.5 |
| Sodium dihydrogen phosphate | | 0.2 | 0.2 | 0.2 |
| HCl/NaOH | | q.s. | q.s. | q.s. |
| Purified water | | q.s. | q.s. | q.s. |
| pH | | 6 | 6 | 6 |
| Remaining | 60° C./ | 96.4 | 24.2 | 69.5 |
| rate (%) | 1 week | | | |
| | 60° C./ | 71.7 | ND | ND |
| | 4 weeks | | | |

ND: no data

As is apparent from Table 1, the formulation of Example 1 maintained significantly high remaining rate at 60° C. over 4 weeks, as compared with the formulations of Comparative Examples 1 and 2. The results revealed that the pharmaceutical composition of the present invention has excellent stability.

### 2. Stability Evaluation Test (2)

An influence of additives and pH in the pharmaceutical composition of the present invention was studied.

### 2-1. Preparation of Test Formulation

In the same manner as in preparation method of Example 1, the formulations of Examples 2 to 24 shown in Tables 2 to 6 were prepared.

### 2-2. Test Procedure

After filling a glass ampule with 5 mL of a test formulation and storing at 60° C. for an optional period, the content of isopropyl (6-{[4-(pyrazol-1-yl)benzyl](pyridin-3-ylsulfonyl)aminomethyl}pyridin-2-ylamino)acetate was determined using high-performance liquid chromatography, and then a remaining rate (%) thereof was calculated.

TABLE 2

| | | Example | Example | Example | Example | Example | Example |
|---|---|---|---|---|---|---|---|
| % (w/v) | | 2 | 3 | 4 | 5 | 6 | 7 |
| Present compound A | | 0.01 | 0.01 | 0.0003 | 0.001 | 0.01 | 0.03 |
| Polyoxy1 35 castor | | 0.8 | 0.8 | 0.5 | 0.8 | 2 | 2 |
| oil | | | | | | | |
| Sodium dihydrogen | | 0.2 | 0.2 | 0.2 | 0.2 | 0.2 | 0.2 |
| phosphate | | | | | | | |
| Sodium edetate | | 0.01 | — | 0.05 | 0.05 | 0.05 | 0.05 |
| hydrate | | | | | | | |
| Glycerol | | 2.3 | 2.3 | — | 2.3 | 2.3 | 2.3 |
| Benzalkonium | | 0.004 | 0.004 | — | 0.004 | 0.004 | 0.00 |
| chloride | | | | | | | |
| HCl/NaOH | | q.s. | q.s. | q.s. | q.s. | q.s. | q.s. |
| Purified water | | q.s. | q.s. | q.s. | q.s. | q.s. | q.s. |
| PH | | 5.8 | 5.8 | 6.0 | 5.8 | 5.8 | 5.8 |
| Remaining | 60° C./ | 94.5 | 88.6 | ND | 93.2 | 94.0 | 94.1 |
| rate (%) | 2 weeks | | | | | | |
| | 60° C./ | 86.2 | 73.0 | 83.2 | 82.2 | 87.1 | 90.9 |
| | 4 weeks | | | | | | |

ND: no data

TABLE 3

| | | Example | Example | Example | Example | Example | Example | Example |
|---|---|---|---|---|---|---|---|---|
| % (w/v) | | 8 | 9 | 10 | 11 | 12 | 13 | 14 |
| Present compound A | | 0.003 | 0.003 | 0.003 | 0.003 | 0.003 | 0.01 | 0.01 |
| Polyoxyl 35 castor | | 0.8 | 0.8 | 0.8 | 0.8 | 0.8 | 0.8 | 0.8 |
| oil | | | | | | | | |
| Sodium dihydrogen | | 0.2 | — | — | — | — | — | — |
| phosphate | | | | | | | | |
| Boric acid | | — | 1 | — | — | — | 1 | — |
| Trisodium | | — | — | 0.2 | — | — | — | — |
| citratedihydrate | | | | | | | | |
| Sodium | | — | — | — | 0.2 | — | — | — |
| acetatetrihydrate | | | | | | | | |
| ε-Aminocaproic | | — | — | — | — | 0.2 | — | — |
| acid | | | | | | | | |
| Trometamol | | — | — | — | — | — | — | 0.9 |
| Sodium edetate | | 0.02 | 0.02 | 0.02 | 0.02 | 0.02 | 0.05 | 0.05 |
| hydrate | | | | | | | | |
| Glycerol | | 2.2 | 1.0 | 2.2 | 2.2 | 2.2 | 1.4 | 0.8 |
| Benzalkonium | | 0.004 | 0.004 | 0.004 | 0.004 | 0.004 | 0.004 | 0.004 |
| chloride | | | | | | | | |
| HCl/ NaOH | | q.s. | q.s. | q.s. | q.s. | q.s. | q.s. | q.s. |
| Purified water | | q.s. | q.s. | q.s. | q.s. | q.s. | q.s. | q.s. |
| pH | | 6.0 | 6.0 | 6.0 | 6.0 | 6.0 | 5.8 | 5.8 |
| Remaining | 60° C./ | 94.6 | 94.0 | 95.1 | 94.3 | 94.4 | 93.1 | 92.6 |
| rate (%) | 2 weeks | | | | | | | |

TABLE 4

| | | Example | Example | Example | Example |
|---|---|---|---|---|---|
| % (w/v) | | 15 | 16 | 17 | 18 |
| Present compound A | | 0.01 | 0.01 | 0.01 | 0.01 |
| Polyoxyl 35 castor | | 0.8 | 0.8 | 0.8 | 0.8 |
| oil | | | | | |
| Sodium dihydrogen | | 0.2 | 0.2 | 0.2 | 0.2 |
| phosphate | | | | | |
| Sodium edetate | | 0.05 | 0.05 | 0.05 | 0.05 |
| hydrate | | | | | |
| Glycerol | | 2.3 | 2.3 | 2.3 | 2.3 |
| Benzalkonium | | 0.002 | 0.008 | 0.004 | 0.004 |
| chloride | | | | | |
| HCl/NaOH | | q.s. | q.s. | q.s. | q.s. |
| Purified water | | q.s. | q.s. | q.s. | q.s. |
| pH | | 5.8 | 5.8 | 5.0 | 6.5 |
| Remaining | 60° C./ | 95.3 | 94.1 | 94.8 | 92.6 |
| rate (%) | 2 weeks | | | | |

TABLE 5

| | | Example | Example | Example | Example |
|---|---|---|---|---|---|
| % (w/v) | | 19 | 20 | 21 | 22 |
| Present compound | | 0.003 | 0.003 | 0.003 | 0.003 |
| A | | | | | |
| Polyoxyl 35 castor | | 0.8 | 0.8 | 0.8 | 0.8 |
| oil | | | | | |
| Boric acid | | 1 | 1 | — | — |
| ε-Aminocaproic | | — | — | 0.2 | 0.2 |
| acid | | | | | |
| Sodium edetate | | 0.02 | 0.02 | 0.02 | 0.02 |
| hydrate | | | | | |
| Glycerol | | 1 | — | 2.3 | — |
| Mannitol | | — | 2 | — | 4.5 |
| Benzalkonium | | 0.0013 | 0.0013 | 0.0013 | 0.0013 |
| chloride | | | | | |
| HCl/NaOH | | q.s. | q.s. | q.s. | q.s. |
| Purified water | | q.s. | q.s. | q.s. | q.s. |
| pH | | 6.0 | 6.0 | 6.0 | 6.0 |
| Remaining | 60° C./ | 93.3 | 93.0 | 93.4 | 93.6 |
| rate (%) | 2 weeks | | | | |

TABLE 6

| % (w/v) | | | Example 23 | Example 24 |
|---|---|---|---|---|
| Present compound A | | | 0.0003 | 0.0003 |
| Polyoxyl 35 castor oil | | | 0.8 | 0.8 |
| Boric acid | | | 1 | 1 |
| Sorbic acid | | | 0.1 | — |
| Sodium edetate hydrate | | | 0.05 | 0.05 |
| Glycerol | | | 1 | 1 |
| Benzalkonium chloride | | | 0.01 | 0.01 |
| HCl/NaOH | | | q.s. | q.s. |
| Purified water | | | q.s. | q.s. |
| pH | | | 6.5 | 6.5 |
| Remaining rate | 60° C. | 2 weeks | 89.1 | 92.5 |

(% )

As is apparent from Tables 2 to 6, the formulations of Examples 2 to 24 maintained a high remaining rate at 60° C. over 2 or 4 weeks.

1. A pharmaceutical composition comprising (i) isopropyl (6-{[4-(pyrazol-1-yl)benzyl](pyridin-3-ylsulfonyl)aminomethyl}pyridin-2-ylamino)acetate or a salt thereof as a sole active ingredient, (ii) citric acid or a salt thereof, (iii) polyoxyethylene castor oil, and (iv) glycerin, wherein
the content of the isopropyl (6-{[4-(pyrazol-1-yl)benzyl](pyridin-3-ylsulfonyl)aminomethyl}pyridin-2-ylamino)acetate or a salt thereof in the pharmaceutical composition is in a range of 0.001 to 0.003% (w/v),
the content of the citric acid or the salt thereof is in a range of 0.1 to 0.2% (w/v),
the content of the polyoxyethylene castor oil in the pharmaceutical composition is in a range of 0.1 to 3% (w/v),
the content of the glycerin is in a range of 2.2 to 3% (w/v),
the polyoxyethylene castor oil is not polyoxyethylene hardened castor oil,
a number of polymerization of ethylene oxide of the polyoxyethylene castor oil is in a range of 5 to 100, and
the pharmaceutical composition does not comprise sorbic acid.

2. The pharmaceutical composition according to claim 1, wherein the polyoxyethylene castor oil includes polyoxyethylene castor oil selected from the group consisting of polyoxyl 5 castor oil, polyoxyl 9 castor oil, polyoxyl 15 castor oil, polyoxyl 35 castor oil, and polyoxyl 40 castor oil.

3. The pharmaceutical composition according to claim 2, wherein the polyoxyethylene castor oil includes polyoxyl 35 castor oil.

4. The pharmaceutical composition according to claim 1, wherein the content of the polyoxyethylene castor oil in the pharmaceutical composition is in a range of 0.5 to 3% (w/v).

5. The pharmaceutical composition according to claim 4, wherein the content of the polyoxyethylene castor oil in the pharmaceutical composition is in a range of 0.8 to 2% (w/v).

6. The pharmaceutical composition according to claim 1, wherein the content of the polyoxyethylene castor oil in the pharmaceutical composition is in a range of 50 to 2,000 parts by mass relative to 1 part by mass of the isopropyl (6-{[4-(pyrazol-1-yl)benzyl](pyridin-3-ylsulfonyl)aminomethyl}pyridin-2-ylamino)acetate or a salt thereof.

7. The pharmaceutical composition according to claim 6, wherein the content of the polyoxyethylene castor oil in the pharmaceutical composition is in a range of 200 to 2,000 parts by mass relative to 1 part by mass of the isopropyl (6-{[4-(pyrazol-1-yl)benzyl](pyridin-3-ylsulfonyl)aminomethyl}pyridin-2-ylamino)acetate or a salt thereof.

8. The pharmaceutical composition according to claim 1, wherein the pharmaceutical composition further comprises edetic acid or a salt thereof.

9. The pharmaceutical composition according to claim 1, wherein the pharmaceutical composition is filled into a container made of polyethylene.

10. The pharmaceutical composition according to claim 1, wherein the pharmaceutical composition is for prevention or treatment of glaucoma or ocular hypertension, or for reduction of intraocular pressure.
